SEQUENCE LISTING (22 sequences)

SEQ ID NO: 1 - protein, 7 aa, Artificial Sequence
  targeting peptide AAV-PR
PRPPSTH

SEQ ID NO: 2 - protein, 7 aa, Artificial Sequence
  targeting peptide AAV-MA
MAEPGAR

SEQ ID NO: 3 - protein, 7 aa, Artificial Sequence
  targeting peptide AAV-SQ
SQDPSTL

SEQ ID NO: 4 - protein, 7 aa, Artificial Sequence
  targeting peptide AAV-ML
MLYADNT

SEQ ID NO: 5 - DNA, 21 bp, Artificial Sequence
  sequence encoding targeting peptide AAV-PR
cctcggccgccgagtacgcat

SEQ ID NO: 6 - DNA, 21 bp, Artificial Sequence
  sequence encoding targeting peptide AAV-MA
atggctgagccgggggctcgt

SEQ ID NO: 7 - DNA, 21 bp, Artificial Sequence
  sequence encoding targeting peptide AAV-SQ
tcgcaggatccgtcgactttg

SEQ ID NO: 8 - DNA, 21 bp, Artificial Sequence
  sequence encoding targeting peptide AAV-ML
atgttgtatgcggataatact

SEQ ID NO: 9 - protein, 4 aa, Artificial Sequence
  targeting peptide consensus sequence
  MISC_FEATURE (1)..(1): X= D or P
XPST

SEQ ID NO: 10 - protein, 7 aa, Artificial Sequence
  targeting peptide
  MISC_FEATURE (1)..(7): either L or D amino acids
HTSPPRP

SEQ ID NO: 11 - protein, 7 aa, Artificial Sequence
  targeting peptide
  MISC_FEATURE (1)..(7): either L or D amino acids
RAGPEAM

SEQ ID NO: 12 - protein, 7 aa, Artificial Sequence
  targeting peptide
  MISC_FEATURE (1)..(7): either L or D amino acid
LTSPDQS

SEQ ID NO: 13 - protein, 7 aa, Artificial Sequence
  targeting peptide
  MISC_FEATURE (1)..(7): either L or D amino acids
TNDAYLM

SEQ ID NO: 14 - protein, 736 aa, Adenoassociated Virus Type 9
  MISC_FEATURE (1)..(736): capsid protein VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 743 aa, Artificial Sequence
  AAV9 VP1 comprising AAV-PR targeting sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPRPPSTHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 743 aa, Artificial Sequence
  AAV9 VP1 comprising AAV-MA targeting sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQMAEPGARAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 743 aa, Artificial Sequence
  AAV9 VP1 comprising AAV-SQ targeting sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSQDPSTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 743 aa, Artificial Sequence
  AAV9 VP1 comprising AAV-ML targeting sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQMLYADNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 7 aa, Artificial Sequence
  retro-inverso targeting peptide
HTSPPRP

SEQ ID NO: 20 - protein, 7 aa, Artificial Sequence
  retro-inverso targeting peptide
RAGPEAM

SEQ ID NO: 21 - protein, 7 aa, Artificial Sequence
  retro-inverso targeting peptide
LTSPDQS

SEQ ID NO: 22 - protein, 7 aa, Artificial Sequence
  retro-inverso targeting peptide
TNDAYLM

(19) United States
(12) Patent Application Publication
(10) Pub. No.: US 2024/0209394 A1
(43) Pub. Date: Jun. 27, 2024

(54) AAV Capsids and Uses Thereof

(71) Applicant: The General Hospital Corporation, Boston, MA (US)

(72) Inventors: Patricia L. Musolino, Boston, MA (US); Casey A. Maguire, Arlington, MA (US); Eloise Hudry, Roxbury Crossing, MA (US); Killian S. Hanlon, Boston, MA (US)

(21) Appl. No.: 18/288,460

(22) PCT Filed: Apr. 27, 2022
(86) PCT No.: PCT/US2022/026608

§ 371 Date: Oct. 26, 2023

Related U.S. Application Data
(60) Provisional application No. 63/180,320, filed on Apr. 27, 2021.

(51) Int. Cl. C12N 15/86; C07K 14/005

(52) U.S. Cl. CPC C12N 15/86; C07K 14/005; C12N 2750/14122; C12N 2750/14143

(57) ABSTRACT

Described herein are capsid peptides that mediated efficient AAV transduction of the brain vasculature, mainly endothelial cells and pericytes, as well as smooth muscle cells, compositions (including AAV), and methods of using the same. Embodiments of the invention encompass AAV capsid proteins comprising an amino acid sequence that comprises at least four, at least five, or at least six contiguous amino acids from the sequence PRPPSTH (SEQ ID NO: 1); MAEPGAR (SEQ ID NO: 2); SQDPSTL (SEQ ID NO: 3); or MLYADNT (SEQ ID NO: 4).

### CLAIM OF PRIORITY

This application claims the benefit of U.S. Provisional Application Ser. No. 63/180,320, filed on Apr. 27, 2021. The entire contents of the foregoing are incorporated herein by reference.

### FEDERALLY SPONSORED RESEARCH OR DEVELOPMENT

This invention was made with Government support under Grant No. DC017117 awarded by the National Institutes of Health. The Government has certain rights in the invention.

### TECHNICAL FIELD

Described herein are capsid peptides that mediated efficient AAV transduction of the brain vasculature, mainly endothelial cells and pericytes, as well as smooth muscle cells, compositions (including AAV), and methods of using the same.

### BACKGROUND

Neurodegeneration and cerebrovascular disease share an underlying microvascular dysfunction and selective transgene delivery to brain endothelium, smooth muscle cells and pericytes may enable gene-targeted therapeutic interventions. The brain vasculature can also serve as a source of secreted therapeutic proteins to neurons and glial cells due to its high density throughout the central nervous system.

### SUMMARY

Two rounds of in vivo selection were performed with an adeno-associated virus serotype 9 (AAV9) capsid scaffold displaying a heptamer peptide library to isolate capsids that traffic to brain after intravenous (iv) delivery. Several capsids were identified that mediated efficient transduction of the brain vasculature, mainly endothelial cells and pericytes, as well as smooth muscle cells, in contrast to parental capsid AAV9, which transduces mainly neurons and astrocytes.

Provided herein are AAV capsid proteins comprising an amino acid sequence that comprises at least four contiguous amino acids from the sequence PRPPSTH (SEQ ID NO:1); MAEPGAR (SEQ ID NO:2); SQDPSTL (SEQ ID NO:3); or MLYADNT (SEQ ID NO:4). In some embodiments, the AAV capsid protein comprises an amino acid sequence that comprises at least five contiguous amino acids from the sequence PRPPSTH (SEQ ID NO:1); MAEPGAR (SEQ ID NO:2); SQDPSTL (SEQ ID NO:3); or MLYADNT (SEQ ID NO:4).

In some embodiments, the AAV capsid protein comprises an amino acid sequence that comprises at least six contiguous amino acids from the sequence PRPPSTH (SEQ ID NO:1); MAEPGAR (SEQ ID NO:2); SQDPSTL (SEQ ID NO:3); or MLYADNT (SEQ ID NO:4).

In some embodiments, the AAV is AAV9.

In some embodiments, the AAV capsid protein comprises AAV9 VP1. In some embodiments, the targeting sequence is inserted in a position corresponding to amino acids 588 and 589 of SEQ ID NO:14.

Also provided herein are nucleic acids encoding the AAV capsid proteins described herein.

Further provided are AAV comprising the capsid proteins described herein. In some embodiments, the AAV further comprises a transgene, preferably a therapeutic transgene.

Additionally, provided herein are targeting sequence comprising [D/P]PST (SEQ ID NO:9). In some embodiments, the targeting sequence comprises at least four contiguous amino acids from the sequence PRPPSTH (SEQ ID NO:1); MAEPGAR (SEQ ID NO:2); SQDPSTL (SEQ ID NO:3); or MLYADNT (SEQ ID NO:4). Further provided are fusion proteins comprising a targeting sequence described herein, and a heterologous sequence. Also provided are AAV capsid proteins comprising the targeting sequences, e.g., wherein the capsid protein comprises AAV9 VP1. In some embodiments, the targeting sequence is inserted in a position corresponding to amino acids 588 and 589 of SEQ ID NO:14. Also provided are nucleic acids encoding the targeting sequences, fusion proteins or AAV capsid proteins, and AAV comprising the capsid proteins. In some embodiments, the AAV further comprises a transgene, preferably a therapeutic transgene. In some embodiments, the transgene encodes Neurturin; Brain Cell Derived Neurotrophic Factor (BDNF); Cerebral dopamine neurotrophic factor (CDNF); mesencephalic astrocyte-derived neural factor (MANF); Vascular endothelial growth factor (VEGF); Glial Cell Derived Neurotrophic Factor (GDNF); Aromatic 1-amino acid decarboxylase (AADC); Tau antibody; Amyloid precursor protein (APP) antibody; type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMHD1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14), or apolipoprotein E epsilon 4 (APOE ε4). Other exemplary transgenes include those described herein.

Also provided herein are methods for delivering a sequence, e.g., a transgene, to a cell; the methods comprise contacting the cell with an AAV as described herein. Also provided are the AAV described herein for use in delivering a sequence to a cell. In some embodiments, the cell is a vascular endothelial cell or smooth muscle cell. In some embodiments, the cell is a pericyte. In some embodiments, the cell is in a living subject, e.g., a mammalian subject.

In some embodiments, the cell is in a tissue selected from the brain, spinal cord, dorsal root ganglion, heart, liver, or smooth muscle, and a combination thereof.

In some embodiments, the subject has a disease that affects the vasculature of the central nervous system, optionally a disease associated with a mutation in type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMHD1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14), or apolipoprotein E epsilon 4 (APOE ε4).

In some embodiments, the subject has a neurodegenerative disease, e.g., Parkinson's disease or Alzheimer's disease.

In some embodiments, the cell is in the brain of a subject, and the AAV is administered by parenteral delivery. In some embodiments, the parenteral delivery is via intravenous, intraarterial, subcutaneous, intraperitoneal, or intramuscular delivery.

In some embodiments, the cell is in the brain of a subject, and the AAV is administered by intravenous delivery.

In some embodiments, the transgene encodes Neurturin; Brain Cell Derived Neurotrophic Factor (BDNF); Cerebral dopamine neurotrophic factor (CDNF); mesencephalic astrocyte-derived neural factor (MANF); Vascular endothelial growth factor (VEGF); Glial Cell Derived Neurotrophic Factor (GDNF); Aromatic 1-amino acid decarboxylase (AADC); Tau antibody; Amyloid precursor protein (APP) antibody; type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMHD1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14), or apolipoprotein E epsilon 4 (APOE ε4). Other exemplary transgenes include those described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-H. AAV-PR-CBA-Cre mediates vasculature-tropic transduction in transgenic Ai9 mice (CAG-floxed-STOP-tdTomato). Mice were injected systemically with AAV-PR-CBA-Cre and sacrificed three weeks later. (A.) Whole-hemisphere image of tdTomato immunofluorescence displaying vasculature transduction by AAV-PR. (B.) Image of the cortical region displaying transduced vasculature and DAPI. (C-D.) High magnification image of the boxed region from (b) showing transduced vasculature. (E) Image from mice injected with AAV-MA-CBA-Cre (4× magnification). (F) Image from mice injected with AAV-MA-CBA-Cre (10× magnification). (G) Image from mice injected with AAV-SQ-CBA-Cre (10× magnification). (H) Image from mice injected with AAV-ML-CBA-Cre (4× magnification).

FIGS. 2A-B. AAV-PR-CBA-GFP mediated vessel transduction decreases over time. Adult male C57BL/6 mice were injected intravenously with 3×10¹⁰ vg/mouse of self-complementary AAV-PR-CBA-GFP and killed at days 5 and 28 for GFP analysis. A. Representative 20× magnification images of coronal 20 μm sections of somatosensory cortex, striatum, and hypothalamus showing transduces vessels. Analyzed areas are depicted by boxes on the brain atlas to the right of the images. B. Quantitation of GFP fluorescent intensity in the three analyzed regions at days 5 and 28. (Cortex, ***p=0.0004; Striatum, *p=0.0135; Hypothalamus, *p=0.0006, respectively).

FIG. 3. AAV genomes in whole brain do not significantly decrease over time. Adult male C57BL/6 mice were injected intravenously with 2.2×10¹⁰ vg/mouse of self-complementary AAV-PR-CBA-GFP and killed at days 5 (n=3) and 28 (n=3) for AAV genome quantitation by Taqman qPCR analysis.

FIG. 4. AAV-PR transduces brain endothelium with high efficiency after systemic injection. Representative images of GFP signal across the somatosensory cortex in 20 μm coronal brain sections of mice injected with AAV-PR-sc-CBA-GFP showing brain endothelial cells transduced (GFP) colocalizing with brain PCAM1 (CD31: red) endothelial marker in capillaries, arterioles and arteries at 5 and 28 days post-injection. At higher magnification (40× boxes) partial colocalization between GFP and CD31 is shown (arrows).

FIG. 5. AAV-PR transduces brain pericytes with high efficiency after systemic injection. Representative images of GFP signal across the somatosensory cortex in 20 μm coronal brain sections of mice injected with AAV-PR-sc-CBA-GFP showing transduced brain vessels (GFP) where pericytes identified by PDGFR-Beta (red) positivity showed significant colocalization at 5 and 28 days post-injection. At higher magnification (40×) partial colocalization between GFP and PDGFR-Beta is shown (arrows).

FIG. 6. Scattered glial and neuronal cells transduced by AAV-PR after systemic injection. Representative images of GFP signal across the somatosensory cortex in 20 μm coronal brain sections of mice injected with AAV-PR-sc-CBA-GFP showing transduced brain endothelium (GFP) and very few astrocyte-shape cells negative for GFAP, MBP and NEUN suggesting perivascular glial precursor cell (yellow arrow) and single pyramidal neuron (red arrow).

FIG. 7. AAV-PR transduction in the liver after systemic injection. Representative images of GFP signal across 20 μm liver sections of mice injected with AAV-PR-sc-CBA-GFP showing transduced hepatocytes (GFP) but not Kupffer, Stellate or vascular cells (endothelium CD31+ or VSMC SMA and SM22+ cells).

FIGS. 8A-C. Illustrative schematic of Experimental Design. A. Two-component system of the library construct. The iTransduce library comprised of different peptide inserts expressed on the capsid (represented by different colors) is injected intravenously (i.v.) into an Ai9 transgenic mouse with a loxP-flanked STOP cassette upstream of the tdTomato reporter gene, inserted into the Gt(ROSA)26Sor locus. AAV capsids able to enter the cell of interest but that do not functionally transduce the cell (no Cre expression) do not turn on tdTomato expression. Capsids that can mediate functional transduction (express Cre) will turn on tdTomato expression. (B) Cells are isolated from the organ of interest (e.g., brain), and transduced cells are sorted for tdTomato expression and optionally cell markers. C. Exemplary images from Ai9 mice administered AAV-CBA-Cre (left) or C57BL.6 mice administered scAAV-CBA-GFP (right). See, Hanlon et al., Mol Ther Methods Clin Dev. 2019 Dec. 13; 15: 320-332.

### DETAILED DESCRIPTION

Many common neurodegenerative diseases involve the brain vasculature including Alzheimer's Disease and Parkinson's Disease. Additionally, there are several rare genetic diseases that affect the vasculature including those associated with mutations in type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMHD1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14); or apolipoprotein E epsilon 4 (APOE ε4).

Gene therapy using AAV vectors has the potential to treat diseases that affect the vasculature. The gold standard in CNS gene therapy, AAV9, which is FDA-approved for treatment of spinal muscular atrophy, mainly transduces neurons and astrocytes with only some limited vasculature transduction. Thus, improvements are needed for therapies targeting the vasculature.

Here, through an in vivo selection process in mice using an AAV9 peptide display library, new capsid targeting peptides were identified. These peptides, when inserted into AAV9 between amino acids 588 and 589, displayed tropism for the CNS vasculature after intravenous systemic delivery. Table 1 lists the peptides.

TABLE 1
Capsid Targeting Peptide Sequences

| | | SEQ | | SEQ |
|---|---|---|---|---|
| | | ID | 21-mer nucleotide | ID |
| Name | Sequence | NO: | sequence | NO: |
| AAV-PR | PRPPSTH | 1 | CCTCGGCCGCCGAGTACGCAT | 5 |
| AAV-MA | MAEPGAR | 2 | ATGGCTGAGCCGGGGGCTCGT | 6 |
| AAV-SQ | SQDPSTL | 3 | TCGCAGGATCCGTCGACTTTG | 7 |
| AAV-ML | MLYADNT | 4 | ATGTTGTATGCGGATAATACT | 8 |

### Targeting Sequences

The present methods identified a number of potential targeting peptides that enhance targeting of CNS vasculature, e.g., when inserted into the capsid of an AAV, e.g., AAV1, AAV2, AAV5, AAV8, or AAV9, or when conjugated to a biological agent, e.g., an antibody or other large biomolecule, either chemically or via expression as a fusion protein.

In some embodiments, the targeting peptides comprise sequences of at least 5 amino acids. In some embodiments, the amino acid sequence comprises at least 4, e.g., 5, 6, or 7 contiguous amino acids of the sequences PRPPSTH (SEQ ID NO:1); MAEPGAR (SEQ ID NO:2); SQDPSTL (SEQ ID NO:3); or MLYADNT (SEQ ID NO:4). In some embodiments, the amino acid sequence comprises at least [D/P]PST (SEQ ID NO:9).

Targeting peptides including L- and D-amino acids can also be used, e.g., L-HTSPPRP (SEQ ID NO:10); L-RAGPEAM (SEQ ID NO:11); L-LTSPDQS (SEQ ID NO:12); L-TNDAYLM (SEQ ID NO: 13); D-HTSPPRP (SEQ ID NO:10); D-RAGPEAM (SEQ ID NO:11); D-LTSPDQS (SEQ ID NO:12); or D-TNDAYLM (SEQ ID NO:13).

Targeting peptides including reversed sequences can also be used, e.g., HTSPPRP (SEQ ID NO:10); RAGPEAM (SEQ ID NO:11); LTSPDQS (SEQ ID NO:12); or TNDAYLM (SEQ ID NO:13).

Targeting peptides disclosed herein can be modified according to the methods known in the art for producing peptidomimetics. See, e.g., Qvit et al., Drug Discov Today. 2017 February; 22(2): 454-462; Farhadi and Hashemian, Drug Des Devel Ther. 2018; 12: 1239-1254; Avan et al., Chem. Soc. Rev., 2014, 43, 3575-3594; Pathak, et al., Indo American Journal of Pharmaceutical Research, 2015. 8; Kazmierski, W. M., ed., Peptidomimetics Protocols, Human Press (Totowa NJ 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746 (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetic include replacing one or more amino acid residues in a peptide with a chemically distinct but recognized functional analog of the amino acid, i.e., an artificial amino acid analog. Artificial amino acid analogs include β-amino acids, β-substituted β-amino acids (“β³-amino acids”), phosphorous analogs of amino acids, such as ∀-amino phosphonic acids and V-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules. Exemplary retro-inverso targeting peptidomimetics include HTSPPRP (SEQ ID NO:19); RAGPEAM (SEQ ID NO:20); LTSPDQS (SEQ ID NO:21); or TNDAYLM (SEQ ID NO:22), wherein the sequences include all D-amino acids. These sequences can be modified, e.g., by biotinylation of the amino terminus and amidation of the carboxy terminus.

### AAVs

Viral vectors for use in the present methods and compositions include recombinant retroviruses, adenovirus, adeno-associated virus, alphavirus, and lentivirus, comprising the targeting peptides described herein and optionally a transgene for expression in a target tissue.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Space for exogenous DNA in AAV is generally limited to an amount of nucleic acid that can physically fit inside the particle. For example, AAV types 1-5 can package up to 6 kb DNA, and in some reports AAV5 has been shown to package up to 8.9 kb DNA. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV3, AAV4, AAV5, AAV6, AV6.2, AAV7, AAV8, rh.8, AAV9, rh.10, rh.39, rh.43 or CSp3; for CNS use, in some embodiments the AAV is AAV1, AAV2, AAV4, AAV5, AAV6, AAV8, or AAV9. Suitable AAV vectors may be designed to accommodate larger amounts of DNA. As one example, AAV9 has been shown to somewhat efficiently cross the blood-brain barrier. Using the present methods, the AAV capsid can be genetically engineered to increase vascular penetration, by insertion of a targeting sequence as described herein into the capsid protein, e.g., into the AAV9 capsid protein VP1 between amino acids 588 and 589.

An exemplary wild type AAV9 capsid protein VP1 (Q6JC40-1) sequence is as follows:

```
  (SEQ ID NO: 14)
                10 20 30 40
MAADGYLPDW LEDNLSEGIR EWWALKPGAP QPKANQQHQD
                50 60 70 80
NARGLVLPGY KYLGPGNGLD KGEPVNAADA AALEHDKAYD
                90 100 110 120
QQLKAGDNPY LKYNHADAEF QERLKEDTSF GGNLGRAVFQ
              130 140 150 160
AKKRLLEPLG LVEEAAKTAP GKKRPVEQSP QEPDSSAGIG
              170 180 190 200
KSGAQPAKKR LNFGQTGDTE SVPDPQPIGE PPAAPSGVGS
              210 220 230 240
LTMASGGGAP VADNNEGADG VGSSSGNWHC DSQWLGDRVI
              250 260 270 280
TTSTRTWALP TYNNHLYKQI SNSTSGGSSN DNAYFGYSTP
              290 300 310 320
WGYFDENRFH CHFSPRDWQR LINNNWGFRP KRLNFKLFNI
              330 340 350 360
QVKEVTDNNG VKTIANNLTS TVQVFTDSDY QLPYVLGSAH
              370 380 390 400
EGCLPPFPAD VFMIPQYGYL TLNDGSQAVG RSSFYCLEYF
              410 420 430 440
PSQMLRTGNN FQFSYEFENV PFHSSYAHSQ SLDRLMNPLI
              450 460 470 480
DQYLYYLSKT INGSGQNQQT LKFSVAGPSN MAVQGRNYIP
              490 500 510 520
GPSYRQQRVS TTVTQNNNSE FAWPGASSWA LNGRNSLMNP
              530 540 550 560
GPAMASHKEG EDRFFPLSGS LIFGKQGTGR DNVDADKVMI
              570 580 590 600
TNEEEIKTTN PVATESYGQV ATNHOSAQAQ AQTGWVQNQG
              610 620 630 640
ILPGMVWQDR DVYLQGPIWA KIPHTDGNFH PSPLMGGFGM
              650 660 670 680
KHPPPQILIK NTPVPADPPT AFNKDKLNSF ITQYSTGQVS
              690 700 710 720
VEIEWELQKE NSKRWNPEIQ YTSNYYKSNN VEFAVNTEGV
              730
YSEPRPIGTR YLTRNL
```

Thus provided herein are AAV that include one or more of the targeting peptide sequences described herein, e.g., an AAV comprising a capsid protein comprising a targeting sequence described herein, e.g., a capsid protein comprising SEQ ID NO:1 wherein a targeting peptide sequence has been inserted into the sequence, e.g., between amino acids 588 and 589.

An exemplary amino acid sequence of AAV9 VP1 comprising the AAV-PR targeting sequence shown in bold lower case is as follows:

(SEQ ID NO: 15)
MAADGYLPDWLEDNLSEGIREWWALKPGAPQPKANQQHQDNARGLVLPGYKYLGPGN
GLDKGEPVNAADAAALEHDKAYDQQLKAGDNPYLKYNHADAEFQERLKEDTSFGGNL
GRAVFQAKKRLLEPLGLVEEAAKTAPGKKRPVEQSPQEPDSSAGIGKSGAQPAKKRL
NFGQTGDTESVPDPQPIGEPPAAPSGVGSLTMASGGGAPVADNNEGADGVGSSSGNW
HCDSQWLGDRVITTSTRTWALPTYNNHLYKQISNSTSGGSSNDNAYFGYSTPWGYFD
FNRFHCHFSPRDWQRLINNNWGFRPKRLNFKLFNIQVKEVTDNNGVKTIANNLTSTV
QVFTDSDYQLPYVLGSAHEGCLPPFPADVEMIPQYGYLTLNDGSQAVGRSSFYCLEY
FPSQMLRTGNNFQFSYEFENVPFHSSYAHSQSLDRLMNPLIDQYLYYLSKTINGSGQ
NQQTLKFSVAGPSNMAVQGRNYIPGPSYRQQRVSTTVTQNNNSEFAWPGASSWALNG
RNSLMNPGPAMASHKEGEDRFFPLSGSLIFGKQGTGRDNVDADKVMITNEEEIKTTN
PVATESYGQVATNHQSAQprppsthAQAQTGWVQNQGILPGMVWQDRDVYLQGPIWA
KIPHTDGNFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPIGTRYLTR
NL

An exemplary amino acid sequence of AAV9 VP1 comprising the AAV-MA targeting sequence (shown in bold, lower case) is as follows:

(SEQ ID NO: 16)
MAADGYLPDWLEDNLSEGIREWWALKPGAPQPKANQQHQDNARGLVLPGYKYLGPGN
GLDKGEPVNAADAAALEHDKAYDQQLKAGDNPYLKYNHADAEFQERLKEDTSEGGNL
GRAVFQAKKRLLEPLGLVEEAAKTAPGKKRPVEQSPQEPDSSAGIGKSGAQPAKKRL
NFGQTGDTESVPDPQPIGEPPAAPSGVGSLTMASGGGAPVADNNEGADGVGSSSGNW
HCDSQWLGDRVITTSTRTWALPTYNNHLYKQISNSTSGGSSNDNAYFGYSTPWGYFD
FNRFHCHFSPRDWQRLINNNWGFRPKRLNFKLFNIQVKEVTDNNGVKTIANNLTSTV
QVFTDSDYQLPYVLGSAHEGCLPPFPADVEMIPQYGYLTLNDGSQAVGRSSFYCLEY
FPSQMLRTGNNFQFSYEFENVPFHSSYAHSQSLDRLMNPLIDQYLYYLSKTINGSGQ
NQQTLKFSVAGPSNMAVQGRNYIPGPSYRQQRVSTTVTQNNNSEFAWPGASSWALNG
RNSLMNPGPAMASHKEGEDRFFPLSGSLIFGKQGTGRDNVDADKVMITNEEEIKTTN
PVATESYGQVATNHQSAQmaepgarAQAQTGWVQNQGILPGMVWQDRDVYLQGPIWA
KIPHTDGNFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPIGTRYLTR
NL

An exemplary amino acid sequence of AAV9 VP1 comprising the AAV-SQ targeting sequence (shown in bold, lower case) is as follows:

(SEQ ID NO: 17)
MAADGYLPDWLEDNLSEGIREWWALKPGAPQPKANQQHQDNARGLVLPGYKYLGPGN
GLDKGEPVNAADAAALEHDKAYDQQLKAGDNPYLKYNHADAEFQERLKEDTSFGGNL
GRAVFQAKKRLLEPLGLVEEAAKTAPGKKRPVEQSPQEPDSSAGIGKSGAQPAKKRL
NFGQTGDTESVPDPQPIGEPPAAPSGVGSLTMASGGGAPVADNNEGADGVGSSSGNW
HCDSQWLGDRVITTSTRTWALPTYNNHLYKQISNSTSGGSSNDNAYFGYSTPWGYFD
FNRFHCHFSPRDWQRLINNNWGFRPKRLNFKLFNIQVKEVTDNNGVKTIANNLTSTV
QVFTDSDYQLPYVLGSAHEGCLPPFPADVEMIPQYGYLTLNDGSQAVGRSSFYCLEY
FPSQMLRTGNNFQFSYEFENVPFHSSYAHSQSLDRLMNPLIDQYLYYLSKTINGSGQ
NQQTLKFSVAGPSNMAVQGRNYIPGPSYRQQRVSTTVTQNNNSEFAWPGASSWALNG
RNSLMNPGPAMASHKEGEDRFFPLSGSLIFGKQGTGRDNVDADKVMITNEEEIKTTN
PVATESYGQVATNHQSAQsqdpstlAQAQTGWVQNQGILPGMVWQDRDVYLQGPIWA
KIPHTDGNFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPIGTRYLTR
NL

An exemplary amino acid sequence of AAV9 VP1 comprising the AAV-ML targeting sequence (shown in bold, lower case) is as follows:

(SEQ ID NO: 18)
MAADGYLPDWLEDNLSEGIREWWALKPGAPQPKANQQHQDNARGLVLPGYKYLGPGN
GLDKGEPVNAADAAALEHDKAYDQQLKAGDNPYLKYNHADAEFQERLKEDTSFGGNL
GRAVFQAKKRLLEPLGLVEEAAKTAPGKKRPVEQSPQEPDSSAGIGKSGAQPAKKRL
NFGQTGDTESVPDPQPIGEPPAAPSGVGSLTMASGGGAPVADNNEGADGVGSSSGNW
HCDSQWLGDRVITTSTRTWALPTYNNHLYKQISNSTSGGSSNDNAYFGYSTPWGYFD
FNRFHCHESPRDWQRLINNNWGFRPKRLNFKLFNIQVKEVTDNNGVKTIANNLTSTV
QVFTDSDYQLPYVLGSAHEGCLPPFPADVEMIPQYGYLTLNDGSQAVGRSSFYCLEY
FPSQMLRTGNNFQFSYEFENVPFHSSYAHSQSLDRLMNPLIDQYLYYLSKTINGSGQ
NQQTLKFSVAGPSNMAVQGRNYIPGPSYRQQRVSTTVTQNNNSEFAWPGASSWALNG
RNSLMNPGPAMASHKEGEDRFFPLSGSLIFGKQGTGRDNVDADKVMITNEEEIKTTN
PVATESYGQVATNHQSAQmlyadntAQAQTGWVQNQGILPGMVWQDRDVYLQGPIWA
KIPHTDGNFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPIGTRYLTR
NL

Exemplary sequences encoding these VPI variants are provided below.

In some embodiments, the AAV also includes a transgene sequence (i.e., a heterologous sequence), e.g., a transgene encoding a therapeutic agent, e.g., as described herein or as known in the art, or a reporter protein, e.g., a fluorescent protein, an enzyme that catalyzes a reaction yielding a detectable product, or a cell surface antigen. The transgene is preferably linked to sequences that promote/drive/regulate expression of the transgene in the target tissue.

Exemplary transgenes for use as therapeutics include transgenes encoding neurturin; brain cell derived neurotrophic factor (BDNF); cerebral dopamine neurotrophic factor (CDNF); mesencephalic astrocyte-derived neural factor (MANF); vascular endothelial growth factor (VEGF); glial cell derived neurotrophic factor (GDNF); aromatic 1-amino acid decarboxylase (AADC); Tau antibody; Amyloid precursor protein (APP) antibody; type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMHD1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14), or apolipoprotein E epsilon 4 (APOE ε4). Other exemplary transgenes include those that encode, e.g., expression of neuronal apoptosis inhibitory protein (NAIP), nerve growth factor (NGF), ciliary neurotrophic factor (CNTF), tyrosine hydroxlase (TH), GTP-cyclohydrolase (GTPCH), amino acid decarboxylase (AADC), aspartoacylase (ASPA), blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-α), transforming growth factor beta (TGF-β), and the like; soluble receptors, such as soluble TNF-α receptors, soluble VEGF receptors, soluble interleukin receptors (e.g., soluble IL-1 receptors and soluble type II IL-1 receptors), soluble gamma/delta T cell receptors, ligand-binding fragments of a soluble receptor, and the like; enzymes, such as α-glucosidase, imiglucarase, β-glucocerebrosidase; enzyme activators, such as tissue plasminogen activator; chemokines, such as IP-10, monokine induced by interferon-gamma (Mig), Groa/IL-8, RANTES, MIP-1α, MIP-10, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2, and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other examples of protein of interest include ciliary neurotrophic factor (CNTF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or nini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

The transgene can also encode an antibody, e.g., an immune checkpoint inhibitory antibody, e.g., to PD-L1, PD-1, CTLA-4 (Cytotoxic T-Lymphocyte-Associated Protein-4; CD152); LAG-3 (Lymphocyte Activation Gene 3; CD223); TIM-3 (T-cell Immunoglobulin domain and Mucin domain 3; HAVCR2); TIGIT (T-cell Immunoreceptor with Ig and ITIM domains); B7-H3 (CD276); VSIR (V-set immunoregulatory receptor, aka VISTA, B7H5, C10orf54); BTLA 30 (B- and T-Lymphocyte Attenuator, CD272); GARP (Glycoprotein A Repetitions; Predominant; PVRIG (PVR related immunoglobulin domain containing); or VTCN1 (Vset domain containing T cell activation inhibitor 1, aka B7-H4).

Other transgenes can include small or inhibitory nucleic acids that alter/reduce expression of a target gene, e.g., siRNA, shRNA, miRNA, antisense oligos, suppressor tRNAs (Wang et al., Nature volume 604, pages 343-348 (2022)) or long non-coding RNAs that alter gene expression (see, e.g., WO2012087983 and US20140142160), or CRISPR Cas9/cas12a and guide RNAs. Genome editing reagents (e.g., a CRISPR Cas nuclease, base editor, or prime editor, and optionally relevant guide RNAs) can also be delivered. For example, a variety of CRISPR systems can be engineered to cut a target sequence of choice. See, e.g., Ledford, Nature. 2021 Sep. 10. doi:10.1038/d41586-021-02461-2. Epub ahead of print; Ramirez-Phillips, AAPS J. 2021 Jun. 2; 23(4):80; Liu et al., Mol Cell. 2022 Jan. 20; 82(2):333-347. In some cases, constructs encoding CRISPR enzymes and gRNAs targeting particular regions are incorporated into an AAV vector as part of the transgene.

The virus can also include one or more sequences that promote expression of a transgene, e.g., one or more promoter sequences; enhancer sequences, e.g., 5′ untranslated region (UTR) or a 3′ UTR; a polyadenylation site; and/or insulator sequences. In some embodiments, the promoter is a vascular endothelial cell-specific promoter, e.g., VE-cadherin promoter, fins-like tyrosine kinase-1 (FLT-1), intercellular adhesion molecule-2 (ICAM-2), a von Willebrand factor (vWF) promoter, a TIE2 promoter, or a synthetic EC-specific promoter (see, e.g., Dai et al., J Virol. 2004 June; 78(12): 6209-6221). In some embodiments, the promoter is a pan-cell type promoter, e.g., a “ubiquitous” promoter that drives expression in most cell types, e.g., cytomegalovirus (CMV) promoter (optionally with the CMV enhancer), chicken beta-actin (CBA) promoter, Rous sarcoma virus (RSV) LTR promoter (optionally with the RSV enhancer), SV40 promoter, dihydrofolate reductase promoter, phosphoglycerol kinase promoter, phosphoglycerol kinase (PGK) promoter, EFlalpha promoter, Ubiquitin C (UBC), B-glucuronidase (GUSB), and CMV immediate/early gene enhancer/CBA promoter; or a steroid promoter or metallothionein promoter. The woodchuck hepatitis virus posttranscriptional response element (WPRE) can also be used.

In some embodiments, the AAV also has one or more additional mutations that increase delivery to the target tissue, e.g., the CNS, or that reduce off-tissue targeting, e.g., mutations that decrease liver delivery when CNS, heart, or muscle delivery is intended (e.g., as described in Pulicherla et al. (2011) Mol Ther 19:1070-1078); or the addition of other targeting peptides, e.g., as described in Chen et al. (2008) Nat Med 15:1215-1218 or Xu et al., (2005) Virology 341:203-214 or U.S. Pat. Nos. 9,102,949; 9,585,971; and US20170166926. See also Gray and Samulski (2011) “Vector design and considerations for CNS applications,” in Gene Vector Design and Application to Treat Nervous System Disorders ed. Glorioso J., editor. (Washington, DC: Society for Neuroscience) 1-9, available at sfn.org/˜/media/SfN/Documents/Short%20Courses/2011%20Short%20Course%20I/2011_SC1_Gray.ashx.

### Targeting Peptides as Tags/Fusions

The targeting peptides described herein can also be used to increase targeting of other (heterologous) molecules to endothelial cells in the CNS vasculature, e.g., by conjugation to the molecule, or by expression as part of a fusion protein, e.g., with an antibody or other large biomolecule. These can include genome editing proteins or complexes (e.g., TALEs, ZFNs, Base editors, and CRISPR RNPs comprising a gene editing protein such as Cas9 or Cas12a, fused to a peptide described herein (e.g., at the N terminus, C terminus, or internally) and a guide RNA), in addition to therapeutic agents or reporters. The fusions/complexes do not comprise any other sequences from Ku70, e.g., comprise heterologous non-Ku70 sequences, and are not present in nature.

### Methods of Use

The methods and compositions described herein can be used to deliver any composition, e.g., a transgene or sequence of interest, to a tissue, e.g., to vasculature of the central nervous system (brain), including endothelial cells and pericytes, as well as to smooth muscle cells of the vasculature. In some embodiments, the methods include delivery to specific brain regions, e.g., cortex, cerebellum, hippocampus, substantia nigra, amygdala.

In some embodiments, the methods and compositions, e.g., AAVs, are used to deliver a nucleic acid sequence to a subject who has a disease, e.g., a disease of the CNS; see, e.g., U.S. Pat. Nos. 9,102,949; 9,585,971; and US20170166926. In some embodiments, the subject has Parkinson's disease, and the vectors are used to deliver neurturin, Brain Cell Derived Neurotrophic Factor (BDNF), Cerebral dopamine neurotrophic factor (CDNF), mesencephalic astrocyte-derived neural factor (MANF), Vascular endothelial growth factor (VEGF), Glial Cell Derived Neurotrophic Factor (GDNF) or Aromatic 1-amino acid decarboxylase (AADC)(See, e.g., Axelsen and Woldbye, J Parkinsons Dis. 2018; 8(2): 195-215; Qin et al., Med Sci Monit. 2022 Mar. 16; 28:e935026; Elabi et al., Sci Rep. 2021 Jan. 13; 11(1):1120; Yu et al., Front Neurosci. 2020 Apr. 29; 14:334). In some embodiments, the subject has Alzheimer's disease, and the vectors are used to deliver Tau antibody or Amyloid precursor protein (APP) antibody (see, e.g., Yang et al, Nature. 2022 March; 603(7903):885-892; Bohannon et al., Cells. 2021 Apr. 14; 10(4):890; Kimbrough et al., Brain. 2015 December; 138(Pt 12):3716-33; Sagare et al., Nat Commun. 2013; 4:2932; Fisher et al., Brain Pathol. 2022 Mar. 14; e13061; Zhang et al., Natl Sci Rev. 2019 November; 6(6):1223-1238; Agyare et al., Mol Pharm. 2013 May 6; 10(5):1557-65). In some embodiments, the subject has a genetic disease that affects the vasculature including those associated with mutations in type IV collagen A1 or A2 (COL4A1/A2)(Vahedi and Alamowitch, Curr Opin Neurol. 2011 February; 24(1):63-8; Mao et al. Dis Model Mech. 2017 Apr. 1; 10(4):475-485); ectonucleotide pyrophosphatase/phosphodiesterase 1 (ENPP1)(Ferreira et al., Genet Med. 2021 February; 23(2):396-407; Maulding et al., Bone. 2021 January; 142:115656); ATP Binding Cassette Subfamily C Member 6 (ABCC6) (Shimada et al., Int J Mol Sci. 2021 Apr. 27; 22(9):4555), three prime repair exonuclease 1 (TREX1)(Hoogeveen et al., AJNR Am J Neuroradiol. 2021 September; 42(9):1604-1609; Rice et al., J Clin Immunol. 2015 April; 35(3):235-43); Forkhead box C1 (FOXC1)(Prasitsak et al., Dev Dyn. 2015 May; 244(5):703-11; French et al., J Clin Invest. 2014 November; 124(11):4877-81); Paired Like Homeodomain 2 (PITX2)(French et al., J Clin Invest. 2014 November; 124(11):4877-81); SAM and HD domain containing deoxynucleoside triphosphate triphosphohydrolase 1 (SAMHD1) (Li et al., Biomed Res Int. 2015; 2015:739586); endoglin (ENG)(Lozano Sinchez et al., J Pers Med. 2022 Mar. 25; 12(4):528); SMAD family member 4 (SMAD4) (Nie et al., Immun Inflamm Dis. 2021 December; 9(4):1306-1320); activin A receptor like type 1 (ACVRL1)(Walsh et al., J Med Case Rep. 2022 Mar. 1; 16(1):99); RAS p21 protein activator 1 (RASA1)(Chen et al., JCI Insight. 2022 Feb. 22; 7(4):e156928); or notch receptor 3 (NOTCH3)(Joutel et al., J Clin Invest. 2000 March; 105(5):597-605; Opherk et al., Hum Mol Genet. 2009 Aug. 1; 18(15):2761-7); HtrA Serine Peptidase 1 (HTRA1) (Shiga et al., Hum Mol Genet. 2011 May 1; 20(9):1800-10); Zinc Finger CCHC-Type Containing 14 (ZCCHCl4)(Traylor et al., Ann Neurol. 2017 March; 81(3):383-394), apolipoprotein E epsilon 4 (APOE E4)(Khera et al., Circulation. 2019 Mar. 26; 139(13):1593-1602). The vectors described herein can be used to deliver a wild type transgene or gene editing reagents (e.g., a CRISPR Cas nuclease, base editor, or prime editor, and relevant guide RNAs) that correct the mutation in the genome of the targeted cells.

The therapeutic agent can be delivered as a nucleic acid, e.g. via a viral vector, wherein the nucleic acid encodes a therapeutic protein or other nucleic acid such as an antisense oligo, siRNA, shRNA, and so on; or as a fusion protein/complex with a targeting peptide as described herein.

### Pharmaceutical Compositions and Methods of Administration

The methods described herein include the use of pharmaceutical compositions comprising the targeting peptides as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intraarterial, subcutaneous, intraperitoneal intramuscular or injection or infusion administration. Delivery can thus be systemic or localized.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser together with instructions for administration.

### EXAMPLES

The invention is further described in the following examples, which do not limit the scope of the invention described in the claims.

### Materials and Methods.

The following materials and methods were used in the Examples below.

Animals: All animal experiments were approved by the Massachusetts General Hospital Subcommittee on Research Animal Care following guidelines set forth by the National Institutes of Health Guide for the Care and Use of Laboratory Animals. We used adult age (8-10 week old) Ai9 (B6.Cg-Gt(ROSA)26Sor^{tm9(CAG-tdTomato)Hze}/J, strain #007909) and C57BL/6J (strain #000664) mice all from The Jackson Laboratory, Bar Harbor, ME.

AAV-PR capsid construction. To create rep cap plasmids encoding AAV9 capsids displaying the AAV-PR peptide (PRPPSTH) for production of vectors encoding a transgene of interest (e.g. GFP or Cre), we digested an AAV9 rep cap plasmid (pAR9) with BsiWI and BaeI which removes a fragment flanking the VP3 amino acid 588 site for peptide sequence insertion. Next we ordered a 997 bp dsDNA fragment from Integrated DNA Technologies (IDT, Coralville, IA), which contains overlapping Gibson homology arms with the BsiWI/BaeI cut AAV9 as well as the 21-mer nucleotide sequence encoding the peptide of interest in frame after amino acid 588 of VP3. Last, we performed Gibson assembly using the Gibson Assembly® Master Mix (NEB, Ipswich, MA) to ligate the peptide-encoding insert into the AAV9 rep cap plasmid. Next we transformed NEB 5-alpha competent E. coli (NEB) with 2 μl of the Gibson assembly and plated transformed cells on LB-Amp agar plates. DNA isolated from selected colonies was sent for Sanger sequencing at the MGH Center for Computational and Integrative Biology DNA Core using a primer that flanked the peptide encoding region to confirm the correct sequence was inserted.

AAV vector production, purification, and titration. For transgene expression studies with AAV-PR vectors we used the following constructs: (1) AAV expression plasmid, pAAV-CBA-NLS-Cres a gift from Miguel Sena-Esteves (UMass Medical Center). This plasmid contains AAV inverted terminal repeats (ITRs) flanking the CBA expression cassette which consists of: a hybrid CMV-IE enhancer/chicken R-actin (CBA) promoter, SV40 nuclear localization signal (NLS), Cre recombinase cDNA, and a bovine growth hormone (BGH) poly A signal sequence. (2) pAAV-sc-CBA-GFP. This construct drives green fluorescent protein (GFP) expression under the hybrid CMV immediate-early/chicken beta actin (CBA) promoter, and was kindly provided by Dr. Miguel Sena-Esteves (UMass Medical Center). AAV-sc-CBA-GFP is a self-complementary (sc) genome.

AAV production was performed as previously described⁶. Briefly, 293T cells were triple transfected (calcium phosphate method) with (1) AAV-PR rep/cap plasmid (2) an adenovirus helper plasmid, pAdΔF6, and (3) ITR-flanked AAV transgene expression plasmid. Cell lysates were harvested 68-72 hr post transfection and purified by ultracentrifugation of an iodixanol density gradient. Iodixanol was removed and buffer exchanged to phosphate buffered saline (PBS) using Zeba desalting columns, 7 kDa molecular weight cutoff (MWCO; Thermo). Vector was concentrated using 2 ml Amicon Ultra 100 kDa MWCO ultrafiltration devices. Vector titers in VG/ml were determined by Taqman qPCR in an ABI Fast 7500 Real-time PCR system (Applied Biosystems) using probes and primers to the BGH poly A sequence and interpolated from a standard curve made with an AAV plasmid. Vectors were pipetted into single-use aliquots and stored at −80° C. until use.

Vector injections in mice. In experiments involving AAV-PR mediated Cre recombination and tdTomato expression in Ai9 mice, we injected adult (both male and female) Ai9 mice systemically via the lateral tail vein with AAV-PR-CBA-Cre vector. At 3-5 weeks post injection animals were killed and brains and other organs processed for cryosectioning and immunofluorescence staining and imaging.

In experiments using AAV-PR as a conventional transgene expression vector, male C57BL/6J mice were injected systemically via the lateral tail vein with AAV-PR-sc-CBA-GFP. Mice were killed at two time points, days 5 and 28 post injection. Brains s were processed for cryosectioning and then immunofluorescence staining and imaging.

Immunofluorescence staining and Microscopy. Sections were dried for twenty minutes at room temperature, and then incubated with PFA for 15 minutes. Then each slide was rinsed three times with PBS for five minutes each. Following this, there was a 10 minute incubation of ice-cold methanol, and then another PBS rinse. Sections were blocked in 5% goat serum and 5% BSA in PBS for 45 minutes. Then incubated overnight at four degrees with primary antibody mixture. Primary antibodies were used as a co-stain as either anti-GFP (G10362; 1:500) and CD31 (BD550274; 1:50) or anti-GFP (ab1218; 1:500) and PDGFr-B (LS C117692; 1:500) based on host species. Slides were rinsed once more with PBS the following day and then incubated with AlexaFlor488 (A32723 or A32731; 1:500) and Alexaflor555 (A-21428 or A-21434; 1:500) for ninety minutes. Slides were treated with ProLong Antifade Mountant with DAPI (P36931). Images were taken using the Zeiss LSM 800 Airyscan confocal microscope located at the Microscopy Core of the Program in Membrane Biology (PMB) at MGH. Widefield fluorescence images were taken at 10×, 20×, 40×, and 63× with standard exposure across all slides. Images were analyzed using ImageJ software (NIH). Reference was made to the Allen Brain Atlas to ensure consistency in image location across different sections and between mouse samples. All image processing and quantification was done using NIH ImageJ software.

### Example 1. AAV-PR Selection and Identification

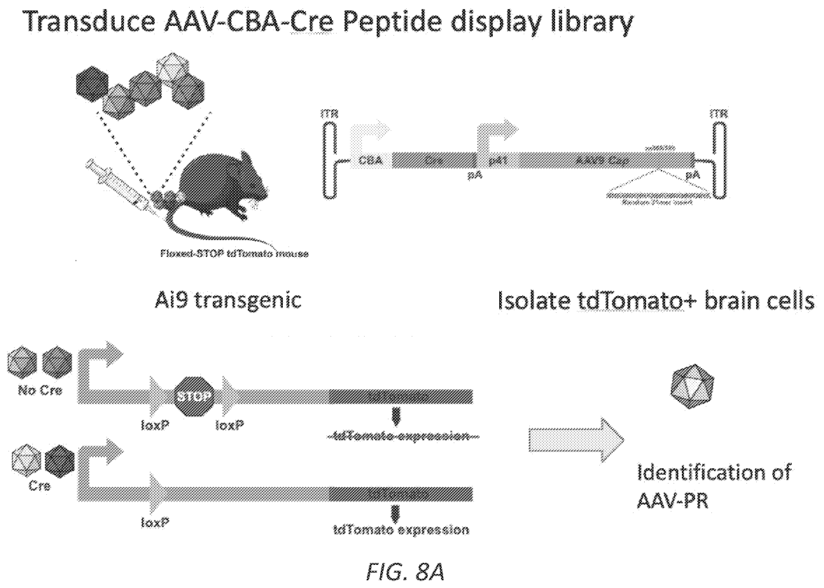

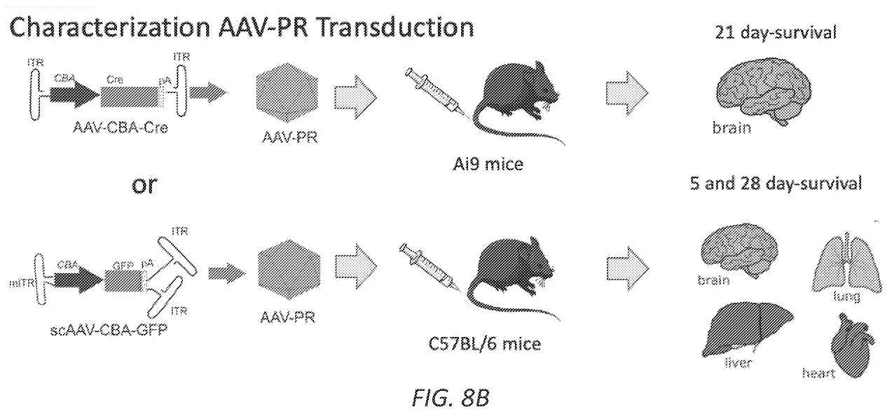

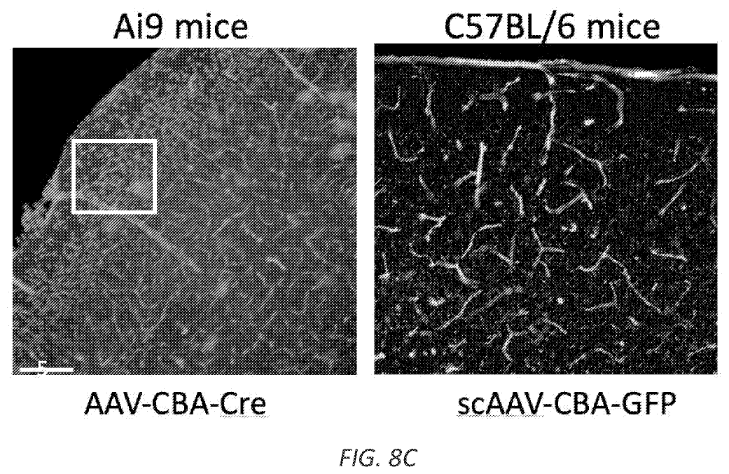

We performed an in vivo selection with an AAV peptide display library to isolate AAV capsids which could transduce brain after systemic injection. The methods are generally described in FIGS. 8A-C; we used our previously described iTransduce library^{7,8} (AAV-CBA-Cre-p41-Cap), which combines a 7-mer peptide display library with a Cre recombinase cassette to couple transgene expression in mice which express a Cre-sensitive fluorescent reporter (Ai9 mice) with rescued peptide encoding sequences. We initially set out with the goal of isolating capsids capable of transducing myeloid derived cells in the brain. We performed two rounds of selection with the capsid library. For the first round of selection, 1.27×10¹¹ vector genomes (vg) of the library was injected in one adult male and one female Ai9 mice via. Three weeks post injection, DNA was extracted from the brain tissue and the CAP region was amplified before re-cloning it back into the AAV plasmid backbone and repackaging for the second round of selection (“brain-enriched capsid library”). For the second round, we used the Cre-cassette to isolate transduction-competent capsids. Two Ai9 females and one male were injected via the tail vein with 1.91×10¹⁰ or 7.64×10¹¹ vg, respectively, of the rescued and re-packaged library. After three weeks, mice were killed, brain cells were dissociated and cells isolated using anti-CD11b/magnetic beads (Magnetic Activated Cell Sorting, MACS, Miltenyi). Next, cells were flow sorted into tdTomato⁺ and tdTomato⁻ fractions. DNA was isolated from each cell pellet as described for the first round and PCR amplification of the peptide insert-encoding region submitted for NGS. From the NGS data, we selected capsid candidates that had a high read frequency in the tdTomato⁺ fraction. DNA was isolated from each cell pellet using the Pico Pure™ DNA Extraction Kit (Thermo Fisher Scientific) and we PCR amplified the cap region containing the insert to determine the peptide profile by next generation sequencing (NGS). From the NGS results, four capsid clones were chosen to evaluate, PRPPSTH (SEQ ID NO:1); MAEPGAR (SEQ ID NO:2); SQDPSTL (SEQ ID NO:3); or MLYADNT (SEQ ID NO:4).

### Example 2. An Engineered Peptide Displaying AAV9 Capsid, AAV-PR, Mediates a Vasculature-Selective Transduction Phenotype in Brain after Intravenous Delivery

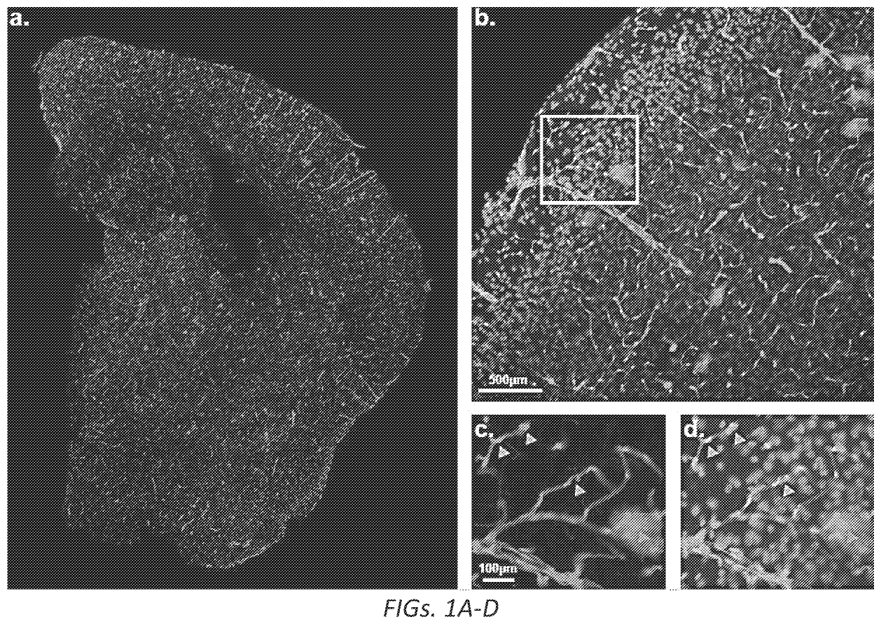

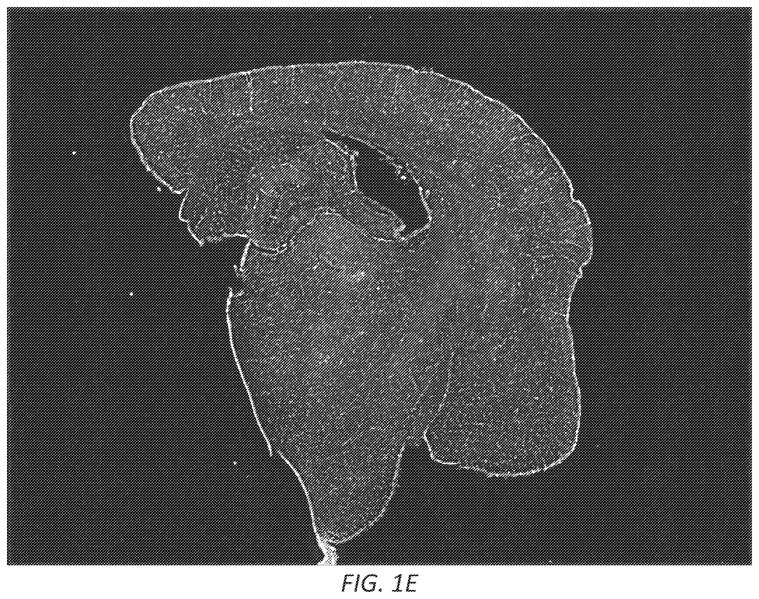

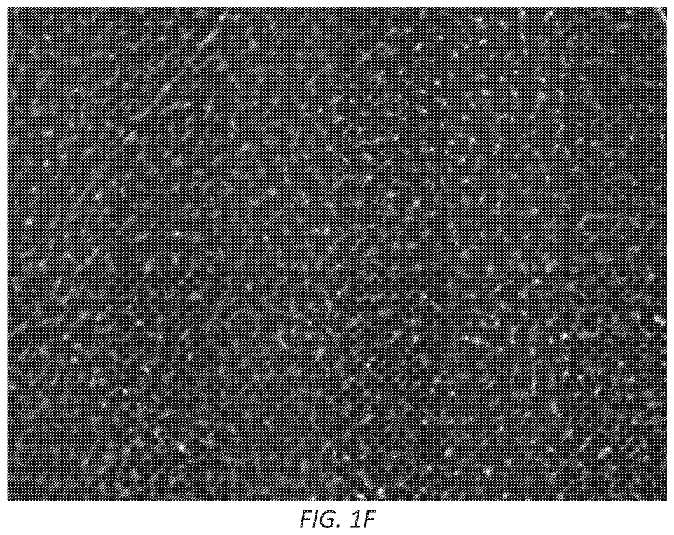

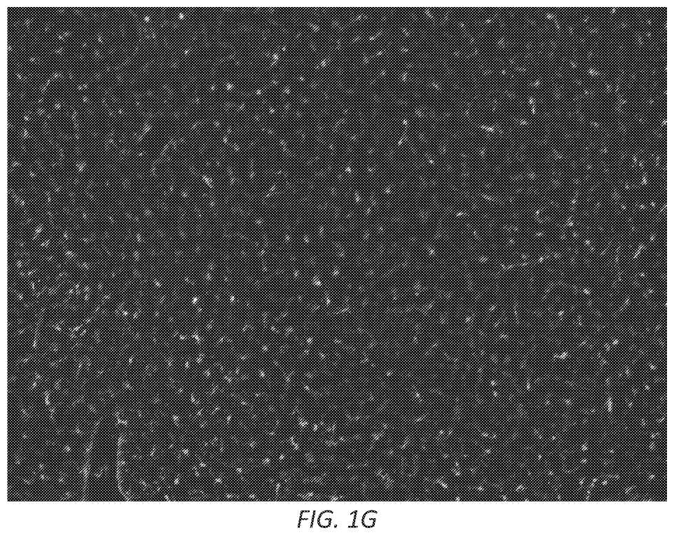

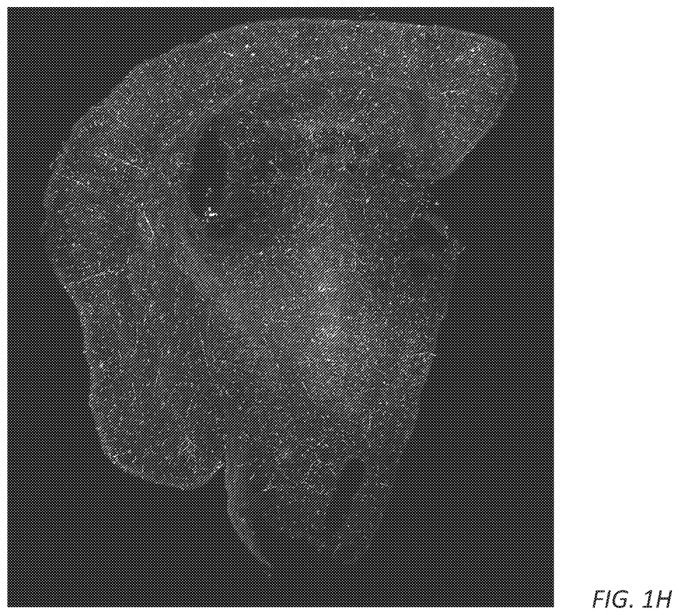

We next tested these new capsids for transduction of the adult murine brain after systemic intravenous delivery. We packaged a single stranded AAV-CBA-Cre genome into each of the candidate capsids and injected this vector (1×10¹² vg/mouse) into the tail vein of adult Ai9 mice which have a Cre-sensitive CAG-floxed-STOP-tdTomato reporter in all cells. Any cells that were successfully transduced by the AAV vector and have Cre-expressed should result in tdTomato expression. Mice were sacrificed three weeks post injection, brains sectioned, and tdTomato detected with immunofluorescence staining. For one capsid, displaying the peptide, PRPPSTH, named AAV-PR, we observed a distinct vasculature immunostaining of tdTomato expression throughout the entire brain (FIGS. 1A-D). This profile is in stark contrast to the tropism of parental AAV9-CBA-Cre in adult Ai9 mice, which mediates transduction of mostly astrocytes and neurons¹. For the other peptides (MAEPGAR (SEQ ID NO:2); SQDPSTL (SEQ ID NO:3); or MLYADNT (SEQ ID NO:4)), the displaying capsids transduced cells within the vasculature as well as cells with neuronal morphology (FIGS. 1E-H).

### Example 3. Time-Dependent Decrease in Brain Vasculature Transduction Cells with IV-Injected AAV-PR Encoding GFP

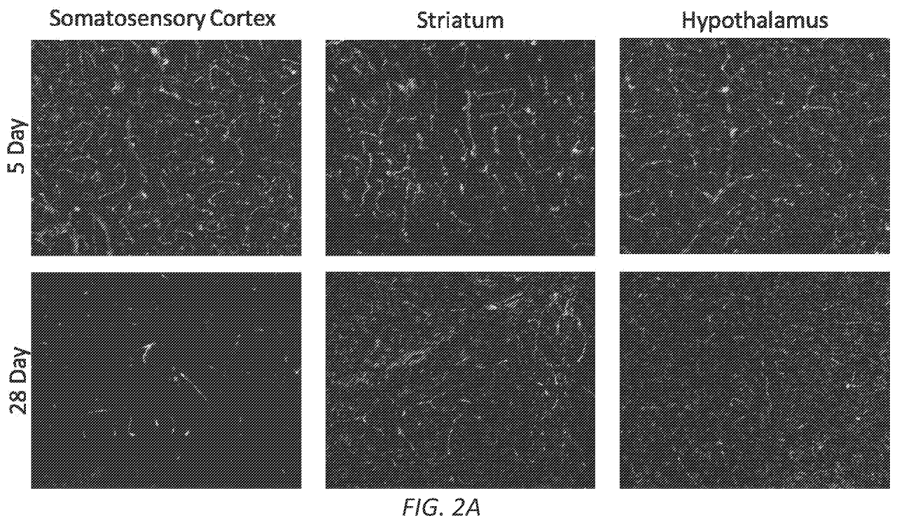

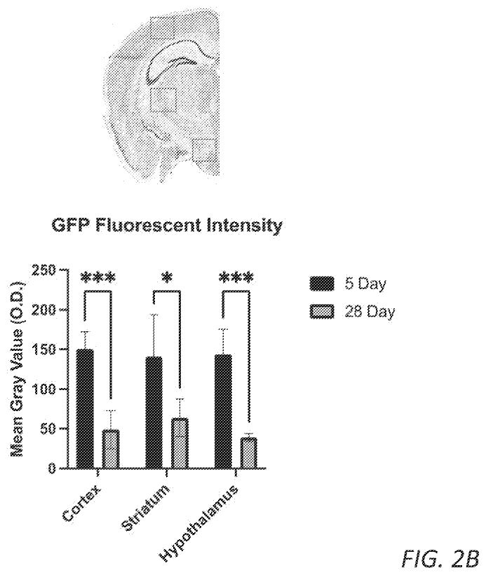

We began our experiments evaluating AAV-PR in a Cre/lox system, to evaluate the “full expression” profile of this capsid. As has been recently demonstrated, traditional fluorescent reporters underestimate transduction events by AAV (e.g. transient expression)², and using the Cre/lox system would enable us to capture any of these transduction events which may be useful for different gene delivery applications, including genome editing. AAV-PR-mediated Cre expression enables permanent genetic modification of the mouse genome resulting in stable tdTomato expression. To test the AAV-PR capsid's transduction profile in a more conventional “gene addition” delivery format, which relies on the stability of the extrachromosomal AAV genome for long-term expression, we packaged a self-complementary (sc) AAV-CBA-GFP genome in the AAV-PR capsid. We injected adult male mice with 3.5×10¹⁰ vg/mouse (approximately 1.4×10¹² vg/kg) and sacrificed mice at day 5 (n=2) and day 28 (n=3) post injection. Brains were sectioned and immunostained for GFP expression. Brains harvested at day 5 revealed transduction primarily in the vasculature as for the AAV-PR-CBA-Cre/Ai9 mouse experiment. This transduction pattern was observed across several brain regions (FIG. 2A). Interestingly, while the vasculature transduction phenotype was maintained at day 28, expression levels were reduced considerably (FIG. 2A). Quantification of GFP levels across three brain regions revealed a significant reduction of around 60% between days 5 and 28 time points (FIG. 2B).

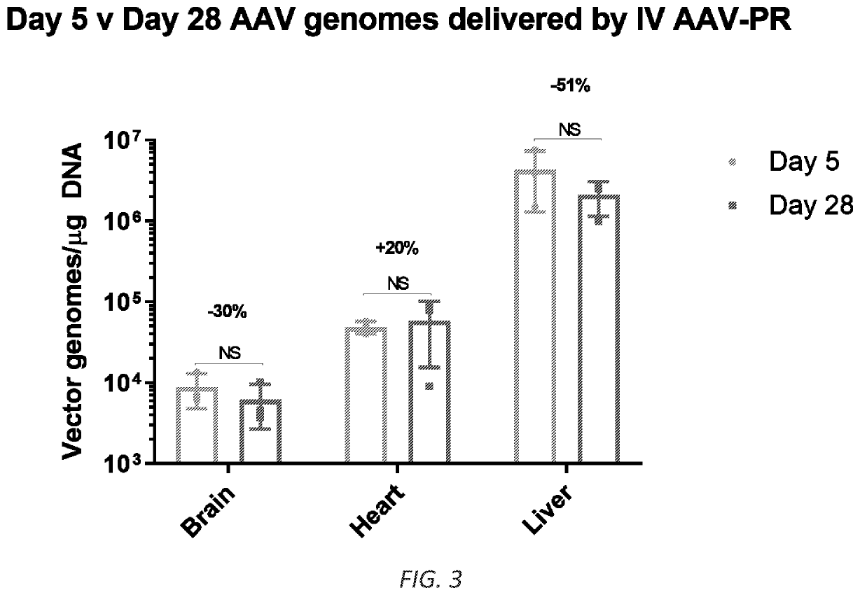

To evaluate the stability of vector genomes in brain, heart and liver, we isolated vector and host genomic DNA from whole tissue at days 5 and 28 post systemic injection of adult male C57BL/6 mice. In brain there was a small 30% decrease in vector genomes between days 5 and d28 (FIG. 3) although this was not statistically significant. In liver there was a 50% decrease and in heart a 20% increase over the same time interval (FIG. 3), although both did not reach statistical significance. The genomes in liver were over 100-fold higher in liver than brain which suggests that AAV-PR capsid is not liver detargeted. These data suggests that there is not a large loss of AAV genomes delivered by AAV-PR over this time period, which may indicate transient activity of the hybrid CBA promoter in endothelial cells.

### Example 4. Characterization of Brain and Peripheral Cell Types Transduced by AAV-PR-CBA-GFP

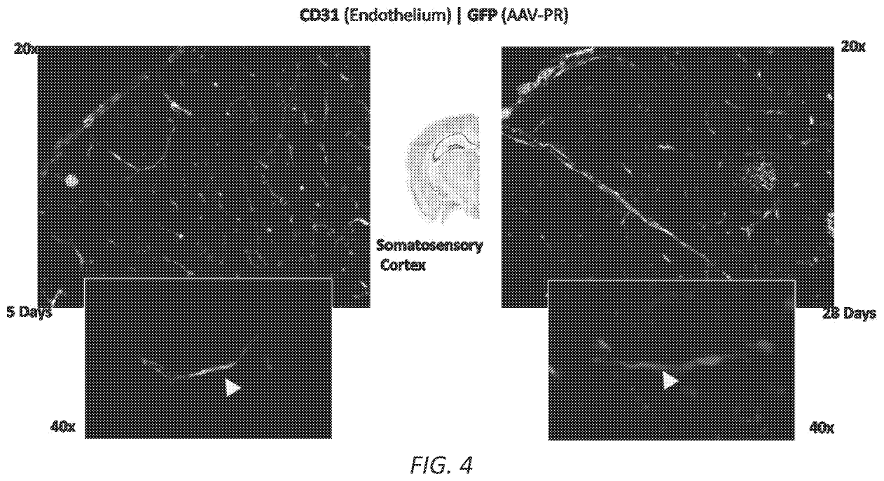

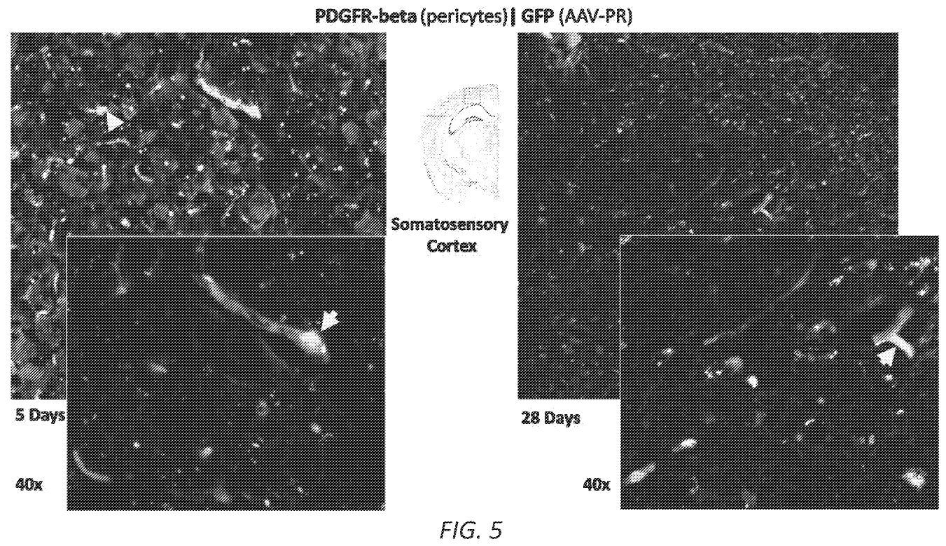

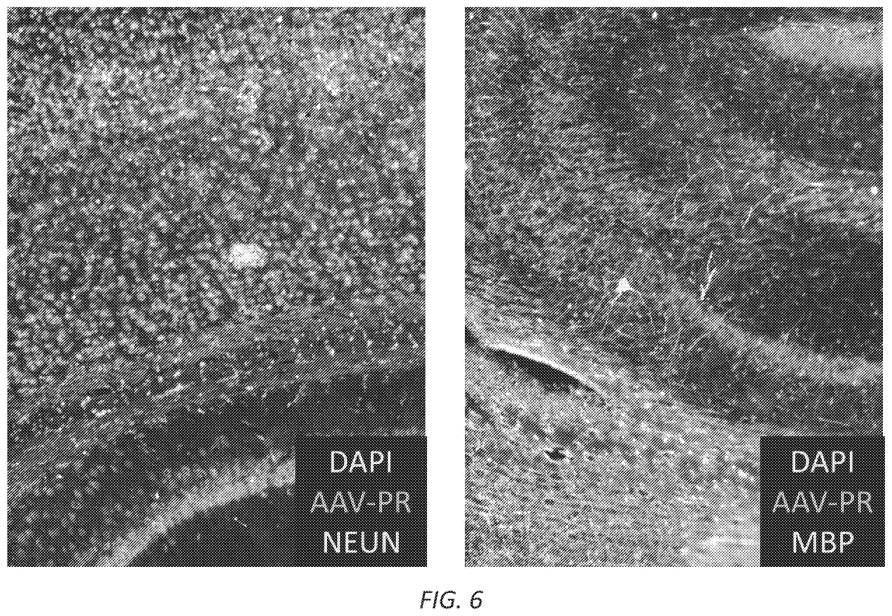

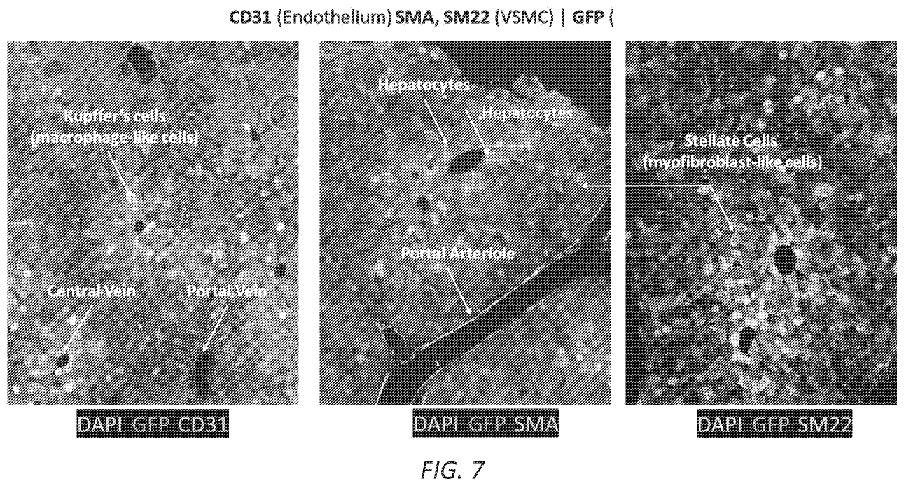

To confirm the vasculature phenotype of transgene expression mediated by AAV-PR-CBA-GFP, brain sections from vector injected mice were immunostained with a variety of cell markers. First, we assessed colocalization of GFP expression with the endothelial marker CD31 at days 5 and 28 post vector injection. At both time points, we readily detected CD31/GFP colocalization as expected (FIG. 4). Next we assessed whether AAV-PR could transduce pericytes. Interestingly, at day 5 we detected up to 50% of pericytes (detected with PDGFR-β staining) were transduced by AAV-PR (FIG. 5). Very sparse detection of GFP positive astrocytes in the somatosensory cortex and neurons n the hippocampus were detected (FIG. 6). As systemically administered AAV vectors often readily transduce the liver, we assessed this organ for GFP expression at day 28. AAV-PR robustly transduced hepatocytes (assessed morphologically) (FIG. 7).

### REFERENCES

1. Prabhakar, S., S. Lule, D. A. H. CC, X. O. Breakefield, and P. S. Cheah, AAV9 transduction mediated by systemic delivery of vector via retro-orbital injection in newborn, neonatal and juvenile mice. Exp Anim, 2021.
2. Lang, J. F., S. A. Toulmin, K. L. Brida, L. C. Eisenlohr, and B. L. Davidson, Standard screening methods underreport AAV-mediated transduction and gene editing. Nat Commun, 2019. 10(1): p. 3415.
3. Xie, J., Q. Xie, H. Zhang, S. L. Ameres, J. H. Hung, Q. Su, R. He, X. Mu, S. Seher Ahmed, S. Park, H. Kato, C. Li, C. Mueller, C. C. Mello, Z. Weng, T. R. Flotte, P. D. Zamore, and G. Gao, MicroRNA-regulated, systemically delivered rAAV9: a step closer to CNS-restricted transgene expression. Mol Ther, 2011. 19(3): p. 526-35.
4. Korbelin, J., G. Dogbevia, S. Michelfelder, D. A. Ridder, A. Hunger, J. Wenzel, H. Seismann, M. Lampe, J. Bannach, M. Pasparakis, J. A. Kleinschmidt, M. Schwaninger, and M. Trepel, A brain microvasculature endothelial cell-specific viral vector with the potential to treat neurovascular and neurological diseases. EMBO Mol Med, 2016. 8(6): p. 609-25.
5. Ravindra Kumar, S., T. F. Miles, X. Chen, D. Brown, T. Dobreva, Q. Huang, X. Ding, Y. Luo, P. H. Einarsson, A. Greenbaum, M. J. Jang, B. E. Deverman, and V. Gradinaru, Multiplexed Cre-dependent selection yields systemic AAVs for targeting distinct brain cell types. Nat Methods, 2020. 17(5): p. 541-550.
6. Ivanchenko, M. V., K. S. Hanlon, M. K. Devine, K. Tenneson, F. Emond, J. F. Lafond, M. A. Kenna, D. P. Corey, and C. A. Maguire, Preclinical testing of AAV9-PHP.B for transgene expression in the non-human primate cochlea. Hear Res, 2020. 394: p. 107930.
7. Hanlon, K. S., J. C. Meltzer, T. Buzhdygan, M. J. Cheng, M. Sena-Esteves, R. E. Bennett, T. P. Sullivan, R. Razmpour, Y. Gong, C. Ng, J. Nammour, D.

Maiz, S. Dujardin, S. H. Ramirez, E. Hudry, and C. A. Maguire, Selection of an Efficient AAV Vector for Robust CNS Transgene Expression. Mol Ther Methods Clin Dev, 2019. 15: p. 320-332.
8. Maguire et al., ENGINEERED ADENO-ASSOCIATED (AAV) VECTORS FOR TRANSGENE EXPRESSION, WO/2020/198737 (January 2020).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An adeno-associated virus (AAV) capsid protein comprising an amino acid sequence that comprises at least four contiguous amino acids from the sequence PRPPSTH (SEQ ID NO:1); MAEPGAR (SEQ ID NO:2); SQDPSTL (SEQ ID NO:3); or MLYADNT (SEQ ID NO: 4).

2. The AAV capsid protein of claim 1, comprising an amino acid sequence that comprises at least five contiguous amino acids from the sequence PRPPSTH (SEQ ID NO:1); MAEPGAR (SEQ ID NO:2); SQDPSTL (SEQ ID NO:3); or MLYADNT (SEQ ID NO:4).

3. The AAV capsid protein of claim 1, comprising an amino acid sequence that comprises at least six contiguous amino acids from the sequence PRPPSTH (SEQ ID NO: 1); MAEPGAR (SEQ ID NO:2); SQDPSTL (SEQ ID NO:3); or MLYADNT (SEQ ID NO4).

4. The AAV capsid protein of claim 1, wherein the AAV is AAV9.

5. The AAV capsid protein of claim 1, comprising AAV9 VP1.

6. The AAV capsid protein of claim 5, wherein the targeting sequence is inserted in a position corresponding to amino acids 588 and 589 of SEQ ID NO:14.

7. A nucleic acid encoding the AAV capsid protein of claim 1.

8. An AAV comprising the capsid protein of claim 1.

9. The AAV of claim 8, further comprising a transgene, preferably a therapeutic transgene.

10. A targeting sequence comprising [D/P]PST (SEQ ID NO:9).

11. A targeting sequence comprising at least four contiguous amino acids from the sequence PRPPSTH (SEQ ID NO: 1); MAEPGAR (SEQ ID NO:2); SQDPSTL (SEQ ID NO:3); or MLYADNT (SEQ ID NO: 4).

12. A fusion protein comprising the targeting sequence of claim 10, and a heterologous sequence.

13. An AAV capsid protein comprising the targeting sequence of claim 10.

14. The AAV capsid protein of claim 13, comprising AAV9 VP1.

15. The AAV capsid protein of claim 14, wherein the targeting sequence is inserted in a position corresponding to amino acids 588 and 589 of SEQ ID NO:14.

16. A nucleic acid encoding the targeting sequence, fusion protein or AAV capsid protein of claim 10.

17. An AAV comprising the capsid protein of claim 13.

18. The AAV of claim 17, further comprising a transgene, preferably a therapeutic transgene.

19. A method of delivering a transgene to a cell, the method comprising contacting the cell with the AAV of claim 9.

20. The method of claim 19, wherein the cell is a vascular endothelial cell or smooth muscle cell.

21. The method of claim 19, wherein the cell is a pericyte.

22. The method of claim 19, wherein the cell is in a living subject.

23. The method of claim 22, wherein the subject is a mammalian subject.

24. The method of claim 19, wherein the cell is in a tissue selected from the brain, spinal cord, dorsal root ganglion, heart, liver, or smooth muscle, and a combination thereof.

25. The method of claim 23, wherein the subject has a disease that affects the vasculature of the central nervous system, optionally a disease associated with a mutation in type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMIHID1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14); or apolipoprotein E epsilon 4 (APOE ε4).

26. The method of claim 23, wherein the subject has a neurodegenerative disease, optionally Parkinson's disease or Alzheimer's disease.

27. The method of any of claim 19, wherein the cell is in the brain of a subject, and the AAV is administered by parenteral delivery.

28. The method of claim 27, wherein the parenteral delivery is via intravenous, intraarterial, subcutaneous, intraperitoneal, or intramuscular delivery.

29. The method of claim 19, wherein the cell is in the brain of a subject, and the AAV is administered by intravenous delivery.

30. The AAV of claim 8, further comprising a transgene, wherein the transgene encodes Neurturin; Brain Cell Derived Neurotrophic Factor (BDNF); Cerebral dopamine neurotrophic factor (CDNF); mesencephalic astrocyte-derived neural factor (MANF); Vascular endothelial growth factor (VEGF); Glial Cell Derived Neurotrophic Factor (GDNF); Aromatic 1-amino acid decarboxylase (AADC); Tau antibody; Amyloid precursor protein (APP) antibody; type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMHD1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14), or apolipoprotein E epsilon 4 (APOE ε4).

31. The method of claim 19, wherein the transgene encodes Neurturin; Brain Cell Derived Neurotrophic Factor (BDNF); Cerebral dopamine neurotrophic factor (CDNF); mesencephalic astrocyte-derived neural factor (MANF); Vascular endothelial growth factor (VEGF); Glial Cell Derived Neurotrophic Factor (GDNF); Aromatic 1-amino acid decarboxylase (AADC); Tau antibody; Amyloid precursor protein (APP) antibody; type IV collagen A1 or A2 (COL4A1/A2); ectonucleotide pyrophosphatase/phosphodiesterase 1 (ENPP1); ATP Binding Cassette Subfamily C Member 6 (ABCC6); three prime repair exonuclease 1 (TREX1); Forkhead box C1 (FOXC1); Paired Like Homeodomain 2 (PITX2); SAM and HD domain containing deoxynucleoside triphosphate triphosphohydrolase 1 (SAMHiD1); endoglin (ENG); SMAD family member 4 (SMAD4); activin A receptor like type 1 (ACVRL1); RAS p21 protein activator 1 (RASA1); notch receptor 3 (NOTCH3); HtrA Serine Peptidase 1 (HTRA1); Zinc Finger CCHC-Type Containing 14 (ZCCHC14), or apolipoprotein E epsilon 4 (APOE ε4)
